(19) United States
(12) Patent Application Publication
(10) Pub. No.: US 2001/0049608 A1
(43) Pub. Date: Dec. 6, 2001

(54) Injection tracking and management system

(76) Inventor: Mark N. Hochman, Lake Success, NY (US)

Correspondence Address: GOTTLIEB RACKMAN & REISMAN PC, 270 MADISON AVENUE, 8TH FLOOR, NEW YORK, NY, 100160601

(21) Appl. No.: 09/765,265

(22) Filed: Jan. 18, 2001

Related U.S. Application Data
(60) Provisional application No. 60/177,902, filed on Jan. 25, 2000.

(51) Int. Cl. G06F 17/60; A61M 31/00; A61M 5/20; G06F 7/00

(52) U.S. Cl. 705/3; 604/156; 700/214

(57) ABSTRACT

An automated drug administering system such an injection device or infusion pump is provided with means for reading information from a container holding the drug. The information is then checked for accuracy before the administration of the drug. Optionally, an ID tag on the patient and/or the health care professional providing the drug may also be scanned and checked. The information thus gathered is sent to another station where it is logged for future use and analyzed.

### RELATED APPLICATION

This application claims priority to provisional application Ser. No. 60/177,902 filed Jan. 25, 2000.

### BACKGROUND OF THE INVENTION

A. Field of Invention

This invention pertains to a system used in a hospital or other health care facility for managing the administration of a drug to a patient using an automated administering device such as an injection device, an infusion device and the like. In a particular embodiment of invention the administration of drugs is monitored and verified to eliminate errors.

B. Description of the Prior art

Recently there has been many reports of rampant medical errors occurring in hospitals and other health care providing facilities. One particular activity occurring at these facilities which is particularly fraught with errors is the provision of drugs (the term ‘drug’ is used herein to refer to any therapeutic substance used to provide therapy to a patient, independently of the delivery or administration method). Typically, a drug is provided to a patient as follows. First, a health professional, normally a physician, orders a drug for a patient by writing a prescription. The prescription is delivered to a pharmacy, where the prescription is transcribed. The drug is then dispensed in a suitable container, i.e., bottle, tube, IV ampule, syringe, etc. As part of this step, an appropriate label is attached to the container. The container is then sent to the side of the patient, and a health practitioner, such as a nurse or a physician, then administers the drug. Studies have shown that mistakes are made at every step of this process. For example, during every step, the wrong drug and the wrong dose, the wrong time, the wrong patient could be specified. In addition allergic reactions and other patient or drug specific problems may also be missed. In fact it was found that the most mistakes occur during the actual administration of the drugs. Of course, it is evident that these mistakes can cause serious injury or even death.

A need for more control and management of drug provision has been recognized in the industry. For example, Becton Dickinson of Franklin Lakes, N.J. 07417 has designed a composite system including a medication management system (referred to as the BD Rx system) to minimize errors during drug delivery, and a specimen collection and drug administration system (referred to as BD.id). The system BD Rx includes an order entry terminal, drug delivery/prescription server and a hand-held unit with printing capability. All the units are electronically coupled to each other so that they can exchange various information. The hand-held unit is used to generate a label for the drug container, including a bar code identifying the patient, and providing other information. However, the system is difficult to apply if the required drug is to be administered by an automated device because such a device adds another level of complexity, and can become another source of error.

### OBJECTIVES AND SUMMARY OF THE INVENTION

An objective of the present invention is to provide a comprehensive injection tracking and management system in which drug administration errors are drastically reduced.

A further objective is to provide a system in which the automated administering of the drugs is monitored to insure the proper patients receive the proper drugs.

A further objective is to provide a system in which the automated administering of a drug is monitored to insure that a specific patient receives the correct drug in the correct dosage and correct injection profile.

A further objective is to provide a injection tracking and management system wherein drug delivery profiles and other associated information descriptive of actual on going procedures for particular patients are automatically logged for further use for that patient and/or other studies.

Other objectives and advantages of the invention will become apparent from the following description.

Briefly, a injection tracking and management system in accordance with this system includes an input used by a physician to provide information for administering a drug to a patient. The device uses this information, and other information from a server storing patient data and from other sources, to generate a prescription in an electronic form. The input device also generates a printed prescription, if required. The transcription is sent to a pharmacy where it is filled. The pharmacy prepares a container with the drug and a label having both standard text and machine-readable characters.

The container with the label is sent to a health care provider, such as a nurse. The health care provider matches the label with the patient and then administers the drug either directly, or using an automated drug administrating device such as an automatic injection device or an infusion pump. Importantly during every step of this process, checks are performed using information from the server with the patient database to insure that the right drug is given to the right patient in the right amount. For this purpose, the automated drug administrating device includes a scanner used to scan the information on the container label and also to identify the patient. The administering device delivers the drug to the patient using a profile dependent on several parameters, such as delivery rate, internal and exit pressure, length of delivery, maximum drug to be delivered, and so on. At the completion of the process, the drug delivery profile is stored with all the other parameters on the server.

### BRIEF DESCRIPTION OF THE DRAWINGS

FIG. 1 shows a block diagram of the injection management system in accordance with this invention;

FIG. 2 shows a block diagram of an injection device used in the injection tracking and management system of FIG. 1;

FIG. 3 shows a flow chart of the operation of the system of FIG. 1; and

FIG. 4 shows a flow chart illustrating the operation of the injection device of FIG. 2.

### DETAILED DESCRIPTION OF THE INVENTION

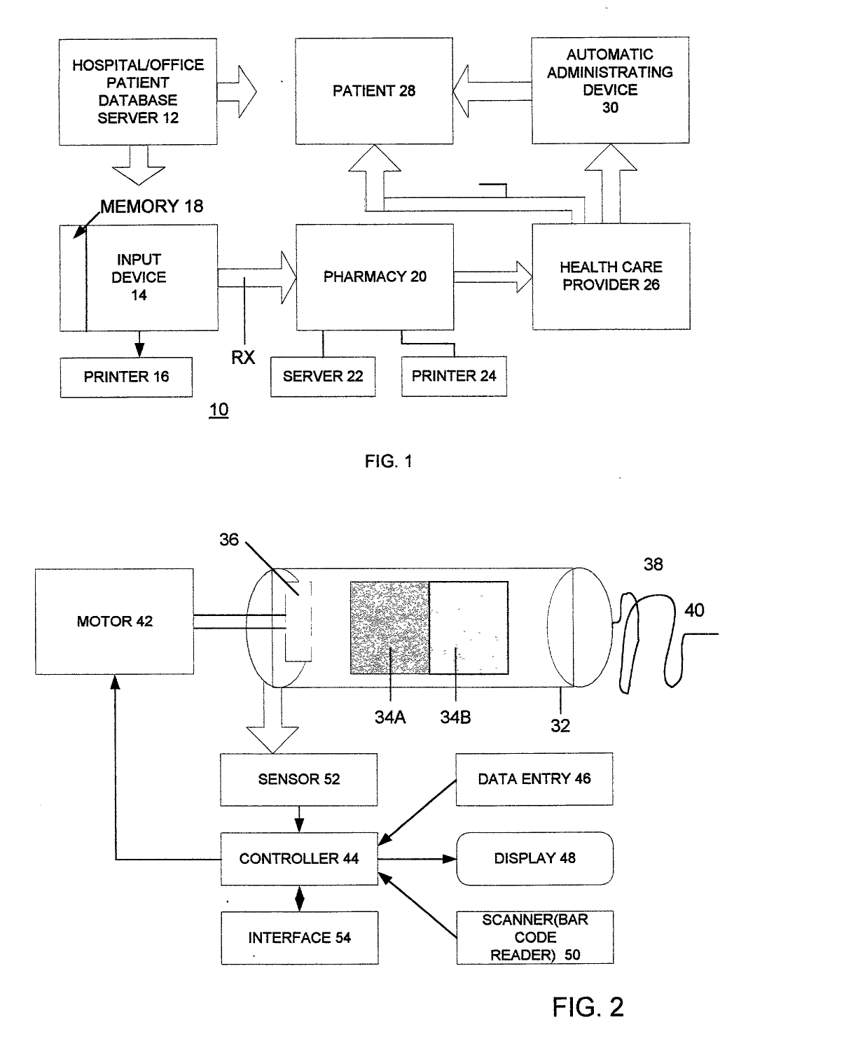

Referring now to FIG. 1, an injection tracking and management system 10 constructed in accordance with this invention includes a patient data base hosted by an appropriate server 12. The server is adapted to store patient data for an office, a wind or department of a hospital, or even a whole hospital. For each patient, the server stores personal information, such as name, address, social security and/or other identifying data. In addition, the medical information of the patient is also stored including a list of various medical procedures or treatments that were provided to the patient, a list of drugs administered, including information on any adverse reactions to any drugs. Importantly, as part of his medical history, the details of the drug administration are also provided. For example, if the drug was an injection, an injection profile is stored identifying the therapeutic agent used for the drug, the volume of drug administered, the rate or rates at which the agent was administered, the exit pressure (discussed more fully below) measured and maintained during the agent injection, the name of the physician who prescribed the agent and the name of the nurse who administered the drug.

System 10 also includes an input device 14. This device 14 may be implemented as a desk top PC, a hand-held device such as a Palm Pilot, and any other device that may be used by a doctor or other health professional to generate a prescription for a patient. The prescription may be generated in a digital form or as a hard copy. The hard copy prescription is generated by a printer 16 attached to the input device 14.

The input device 13 is also associated with a memory 18 which is used to store a drug data base. This drug data base contains a comprehensive listing of various drugs, their indicated usage, side effects, and other information released by the drug manufacturer. The memory 18 can be incorporated into the device 14 or can be located remotely and accessed by device 14 as required by using a wired or wireless Internet or Intranet connection. The device 14 also provides access by similar means to the patient data base 12 so that the physician writing a prescription can access information regarding the patient.

The printer 16 is used to print the prescription (if necessary) in a hard copy format. Preferably, the printed prescription includes plain text readable by a person and indicating the name of the patient, the prescribed drug, the method of administering the drug, and other similar information. In addition, the hard copy prescription also includes a machine-readable portion which contains the same information as the plain text. This portion may consist of bar codes. A magnetic strip and other similar machine-readable characters.

The electronic or printed prescription is then provided to the pharmacy 20. The pharmacy has its own server 22. Once a prescription is received, it is filled manually by a pharmacist. The pharmacist uses the server 22 to access the data base 12 and the pharmacy inventory (also stored in the server 22) to insure that (1) the patient information on the prescription is correct and (2) that the requested drug is available. Warnings and other information regarding the requested drug is also retrieved from server 22. If the patient information is found to be correct. A printer 24 is provided at the pharmacy to print a label LX which contains all the information on the prescription, and additional information about the respective drug, including the manner in which to be administered, and specific information such as the lot number of the drug. The information is stored on the label in two forms: in plain text so that it can be read by a clinician, and in machine-readable form (such as a bar code).

The pharmacist affixes the label LX to the container CX and pass the container onto the health care provider 26. The health care provider 26 reads label LX to identify the patient 28 for whom the drug was selected. If the drug is to be taken orally or by other mechanical means, the health care provider 26 takes the necessary actions.

If the drug is to be administered subcutaneously using an automated device 30 such as an automatic injection device, or an infusion pump, then the device itself is programmed to check the information on the label. Preferably, the automatic device is provided with a reader adapted to read the information on the label. Additional checks may also be performed to insure that the drug is properly administered by the device 30 as described more fully below.

Just to complete the cycle, once the health provider 26 administers the drug to patient 28, he may then update the data base server indicating what drug has been administered to the patient, together with specifics on the drug and/or administration process itself.

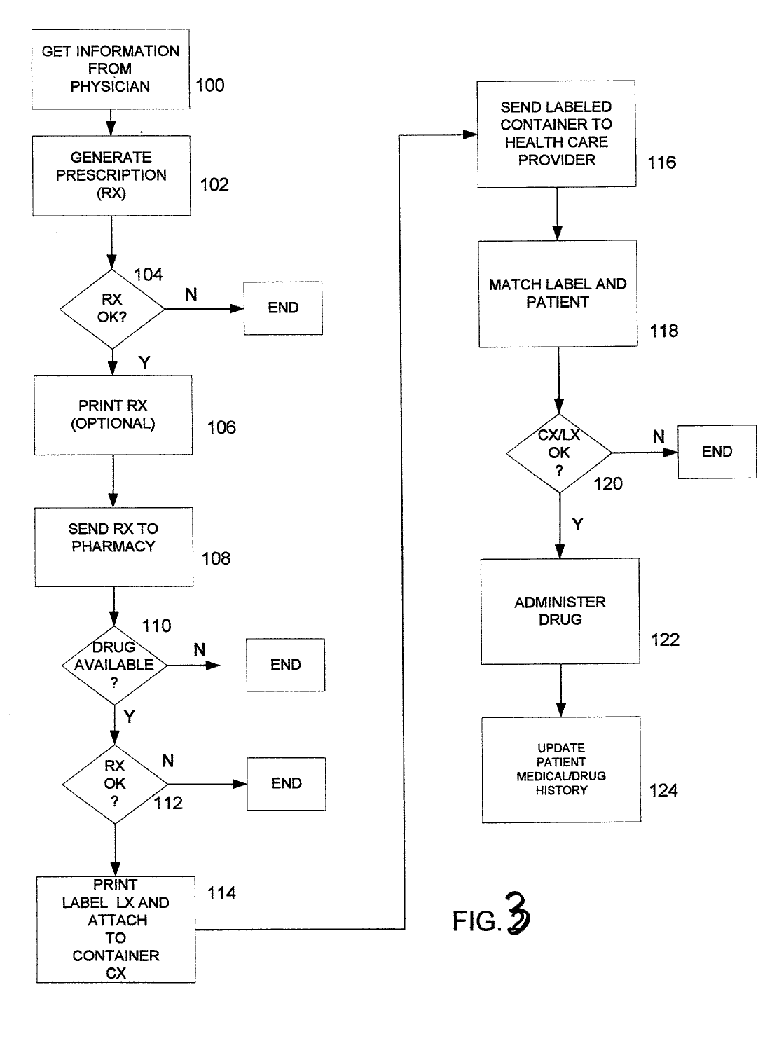

Referring now to FIG. 2, an automatic injection device 30A adapted to perform the function described for device 30 in FIG. 1. FIG. 2 shows a syringe 32 with a label LX. As discussed above, the label LX includes two portions: a portion 34A which contains information in plain text form and a portion 34B which contains substantially the same information in a machine readable form.

A piston 36 is disposed within the syringe 32 so that it can reciprocate along its longitudinal axis thereby causing liquid in the syringe to be selectively expelled and aspirated. A tube 38 is attached to the other end of the syringe. The tube 38 is terminated with a needle 40. The piston 36 is coupled to a motor 42 to drive the syringe. The motor 42 is controlled by a controller 44. The controller is also connected to a data entry device 46, such as a keyboard 46, a display 48, a scanner 50 or other means such as a bar code reader adapted to recognize the machine-readable portion 34B of the label LX. The controller 44 is further connected to a sensor 52. The sensor 52 is adapted to sense a force or pressure developed in the device as the liquid is expelled from the syringe CX and into the patient. An injection device of the type shown in FIG. 2 is disclosed in commonly assigned application Ser. No. 09/201,464 filed Nov. 30, 1998, entitled PRESSURE/FORCE COMPUTER CONTROLLED DRUG DELIVERY SYSTEM now U.S. Pat. No. ______ and incorporated herein by reference, however that device does not have a scanner.

As discussed in detail in that application, the sensor 52 may be used to monitor the force generated by the pump, the pressure developing within the syringe, or, by taking into consideration the size and other characteristics of the syringe the tube 38 and needle 40, the exit pressure at which the fluid is expelled from the needle 40 may also be determined.

In the present invention, the device 30A is also provided with an interface 54 used to exchange data with the server 12, and/or any of the other components of the system 10. Using this interface, the device 30A obtains information about the patient, a prescription, other information required for its programming, and can also send information to the other system components, including an update of the patient history, once a particular drug has been administered.

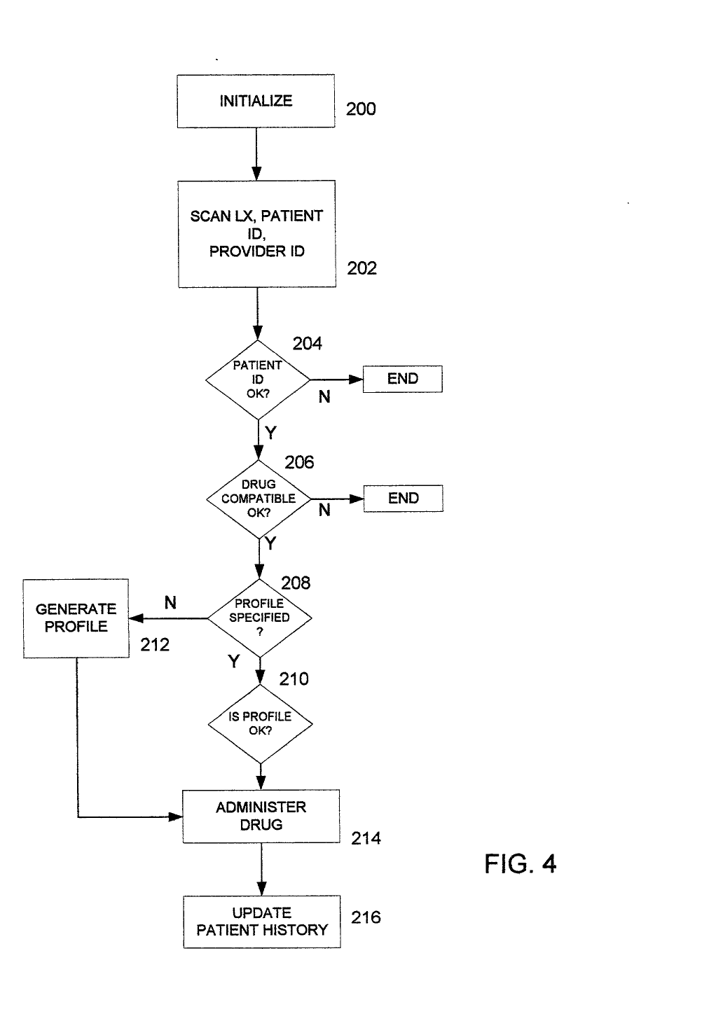

In order to provide a better understanding of the present invention, reference is now made to the flow charts of FIGS. 3 and 4 which illustrate the operation of the system 10 and device 30A. Starting with FIG. 3, in step 100 the input device 14 receives information from the physician identifying a particular patient 28 of the subject system 10 and a drug that has to be administered to this patient. As the information is entered, the physician can check side effects, contra-indications for the drug and other medical data stored in memory 18. Once the drug and details of its administration have been selected, the input device 14 generates a prescription RX (step 102). In step 104 the input device 14 contacts the server 12 to determine if the prescription refers to a patient entered in its database. The device 14 also checks (step 106) whether the specified patient is clinically allowed to receive the designated drug (i.e., he is not allergic to the designated drug, has not been administered an excessive dosage, etc.). In step 106 a hard copy of the prescription of the prescription RX is printed (if necessary), as discussed above.

In step 108 the prescription RX is sent to the pharmacy 20. In step 110 the pharmacy 20 checks if the designated drug is available. In step 112 the pharmacy checks whether the prescription RX is correct. As part of this check, the pharmacist (through server 22) can automatically or manual checks if the patient on the prescription is a patient in the database of server 12 and whether the prescribed drug, dosage and prescribed method of administration are correct.

Next, in step 114 a label LX is printed by printer 24 and attached to a container CX, as discussed above.

Next, in step 116 the container CX with the correct label LX is sent to a health care provider 26. In step 118 the health care provider 26 matches the label on the container with the patient and checks if the drug and it administration is correct (step 120). In step 122 the drug is administered to the patient either manually by the health care provider, or through an automatic administrating device 30. In step 124 the health care provider, or the device 30 updates the medical/drug history of the patient 28 in the database of server 12.

Referring now to FIG. 4, if a drug is to be administered by an automatic means, such as the injection device 30A of FIG. 2, then the following operation takes place. In step 200 the health care provider (or other personnel) mounts the container CX with label LX into the machine and initializes the device 30A. As part of this initialization, and before or after the container CX is mounted on the device 30A, its label CX is scanned using the scanner 50 (step 202). Next, in step 204 the patient's ID is checked. For example, if the patient 28 is wearing a bracelet with some scannable indicia, then it can be scanned using scanner 50.

Next, in step 206 the compatibility issues between the patient and the drug are checked again. In step 208 a check is performed by the device 30A to determine if a profile for the drug administration has been provided as part of the prescription on label LX or by the health care provider. Such a profile may include the total amount of the drug to be administered, the rate at which the drug is to be administered, threshold parameters, such as peak rate, peak internal or exit pressure, etc. If a complete profile has not been specified, then the device may specify a profile in step 210, using the past drug history of the patient, past drug history of other patients, recommendations by the drug manufacturer, etc.

If a profile has been specified then this profile is checked in step 214 to insure that the profile meets certain qualifications.

Next in step 214 the drug is administered and in step 216, after the drug administration is completed, the device 30A sends a message to the server 12 to update the medical/drug history of the patient.

In this manner, at every step of the way from the time the physician provides the information that generates the prescription until the actual administration of the corresponding drug, many checks are performed to make sure the right patient gets the right medicine in the correct dosage under the right conditions. At the end of the administration, the details of the procedure are immediately logged in the database so that they are readily available. Every time a check indicates an abnormal result, for example, in steps 104, 110, 112, 120, 204, 206, 208, 212, the process is halted.

Importantly, during, or at the completion of its operation, the automated administration device sends a complete drug deliver profile of the respective procedure including all, or at least some of the following information:

Patient Name

Patient ID

Patient's Physician

Attending Physician

Prescription

Dispensing pharmacist

Drug Identification by name, lot and manufacturing date

Identification of Health care provider administering drug delivery to patient

Identification of automated administering device

Drug delivery profile (time dependent volume, rate and/or pressure characteristics)

This information is then available from the server to physicians for future therapy for the patient and/or other statistical analyses regarding the efficacy of the drug and other matters.

The invention has been described in terms of an injection device. Other devices which may be adapted to provide the checks described herein include infusion pumps, and injection devices.

Obviously numerous modifications may be made to this invention without departing from its scope as defined in the appended claims.

I claim:

1. An automated administering device adapted to deliver a drug from a container having a machine readable label to a patient comprising:
a holder adapted to hold said controller;
a motor coupled to said holder and adapted to deliver said drug from said container to the patient;
a controller coupled to said motor and arranged to selectively activate said motor; and
a scanner coupled to said controller and arranged to read identification information associated with said drug from said container, said controller operating in accordance with said information.

2. The device of claim 1 further comprising an interface adapted to receive further information regarding the drug delivery, said controller being adapted to compare said identification information with said further information for determining if the administration of the drug is allowable.

3. The apparatus of claim 2 wherein said controller is adapted to receive patient information.

4. The apparatus of claim 3 wherein said controller is adapted to receive said patient information through said scanner.

5. The apparatus of claim 2 wherein said controller is adapted to receive information associated with the person operating the device.

6. A method of administering a drug to a patient using an automated drug administering device with means adapted to obtain electronic information comprising the steps of:
scanning information from a container holding the drug to be administered, said information including one of a patient's identity, the drug identity and a drug delivery profile;
checking said information to determine if said information is correct; and
administering the drug only of during said checking step said automated drug administering device determines that said information is correct.

7. The method of claim 6 further comprising the step of contacting a remote site and obtaining data related to said drug administration and said checking step.

8. The method of claim 6 further comprising scanning electronically a tag associated with the patient to determine the patient identity and matching said patient identity with the information from said container.

9. The method of claim 6 wherein said container is provided by a health providing professional having an identifying tag, further comprising scanning said identifying tag and logging an attendant information based on said identifying tag.

10. The method of claim 6 further comprising storing an applied profile indicative of the administering of said drug to the patient.

11. The method of claim 10 further comprising transmitting said applied profile to a remote location.

12. The method of claim 11 wherein said applied profile includes one of patient information, drug information, attendant information.
